(19) United States
(12) Patent Application Publication
(10) Pub. No.: US 2025/0099811 A1
(43) Pub. Date: Mar. 27, 2025

(54) SYSTEM AND METHOD FOR HAND REHABILITATION

(71) Applicant: Moshe SHOHAM, Hoshaya (IL)

(72) Inventor: Moshe SHOHAM, Hoshaya (IL)

(21) Appl. No.: 18/728,904

(22) PCT Filed: Jan. 15, 2023
(86) PCT No.: PCT/IL2023/050044

§ 371 Date: Jul. 15, 2024

Related U.S. Application Data
(60) Provisional application No. 63/299,985, filed on Jan. 16, 2022.

(51) Int. Cl. A63B 24/00; A63B 23/16; G16H 20/30

(52) U.S. Cl. CPC A63B 24/0062; A63B 23/16; G16H 20/30; A63B 2220/58; A63B 2220/803; A63B 2220/807

(57) ABSTRACT

A novel system for hand rehabilitation comprising a combination of finger position devices and velocity measurement devices, providing the exact spatial location and orientation of the fingers and the thumb, enabling measurement of movements of the hand, including position and rotations. The movements and positions outputs are monitored while the subject is using the hand to perform actions on an object with known physical, geometric and kinematic parameters, such as dimensions, weights and stiffnesses. By knowing both the hand's motion and the physical, geometrical and kinematic parameters of the above-mentioned tools, the system can calculate the forces exerted by the patient's hand, measure motion ranges and track the rehabilitation process without a need for using force sensors or sensors inside the tools. Computer processing and gaming applications are used to encourage patient rehabilitation involvement and motivation and to assess the rehabilitation process.

### FIELD OF THE INVENTION

The present disclosure describes technology related to the field of hand rehabilitation, especially without the need for force sensors attached to the digits of the subject's hand or on therapy accessories.

### BACKGROUND

The hand rehabilitation process often requires repetitive motions of the hand and exertion of forces by hand muscles including muscles of the fingers and the thumb. Multiple systems for performing this process exist in the form of wearable gloves or wearable sensors that enable both passive and active exercise, as well as 3-D localization of the hand. Some such commercially available systems include, for example, the “Sinfonia” system available from Gloreha—Idrogenet srl, of Lumezzane B S, Italy the “Hand Tutor” system, available from Meditouch Ltd. of Netanya, Israel, and the “Amadeo” system, available from TyroMotion Inc. of Marietta, GA, USA. These devices can measure hand and finger movements, and, some of them may use force sensors to measure the forces exerted by the hand, the force sensors being mounted on the hand parts, or sometimes on the therapy accessories. A combination of these two measurements is sufficient for enabling the analyzing of the muscular effects which achieve the motion of the hand, and hence the progress of the rehabilitation process. However, the existing devices are limited in their ability to measure rotational as well as lateral movements together with the forces. In addition, some make use of built-in therapy sensors inside the wearable glove, and such sensors, or at least their electronic driver and measurement circuits, even if in chip form, make the systems more complex and sometimes relatively costly. Furthermore, the gloves may sometimes be somewhat awkward to wear, and may have an unnatural feel to the person undergoing the rehabilitation.

Examples of previously described methods and devices of hand rehabilitation, which may be considered to represent the present state of the art, can be found in the following patent applications or patents:

U.S. Pat. No. 10,299,738B2, WO2014186537A1, KR102239670B1, ES2351143B1, EP3583487A1, U.S. Pat. No. 10,758,158B2, US20130072829A1, WO2020226518A1, U.S. Pat. No. 10,849,815B2 and U.S. Pat. No. 9,891,718B2.

The disclosures of each of the publications mentioned in this section and in other sections of the specification, are hereby incorporated by reference, each in its entirety.

### SUMMARY

The present disclosure attempts to provide novel systems and methods that overcome at least some of the disadvantages of prior art systems and methods. The present disclosure describes a novel hand rehabilitation system which differs from previously available systems in that it does not require force sensors to measure forces exerted by the patient's hand or hand parts on the object being handled, whether a device or a hand exercising physiotherapy tool or an occupational therapy object. Motion ranges are detected using either wearable location and orientation sensors, whose signals are remotely accessed by wireless, or, by imaging the hand parts themselves or wearable referencing marks on the hand parts, by a remote imaging camera, and determining the location and orientation of the hand parts such as by image processing of the images. The forces exerted by the particular hand muscles or index being tested or exercised are determined by a knowledge of the extent of motion achieved by the index when working on an occupational therapy tool or a similar device whose physical, kinematic and geometrical parameters are known. The location sensors, whether wearable, remote, or camera based, provide the exact location and tracking of hand and finger movements. The wearable sensors are wireless-based, enabling free motion of the hand. The sensors are positioned on phalanges of the hand. In one embodiment, the sensors can be placed on the hand using a light-weight wearable elastic glove. In a second embodiment, they are placed on the fingers as individual wearables. Since force sensors are generally more complex and bulky components than position sensors, the avoidance of their use in the present application, greatly simplifies the system, and ensures less encumbrance to the user, as well as enabling measurement of forces in all directions, including adductions and abductions.

In another embodiment, the finger locations may be determined by a system located remotely from the hand, and detecting the exact location of the finger, without the need to apply any wearable device on the fingers. The remote locating system can be based on visible or infra-red signals and a video image processing system, though other detection methods may also be used. A combination of the above two embodiments is also possible, in that the remote imaging system images highly visible wearable markers to increase the accuracy of the remote imaging process.

The spatial information from the sensors is analyzed by a signal reception and processing system, which receives inputs from the abovementioned sensors, regarding the hand's or the digits' location and hence, also their motion, and sends the information to the system controller.

The system makes use of occupational therapy tools or toys such as, but not limited to, separable magnets, large and small plastic beads, theraputty, cloth pins, springs, plastic bottles, sponge balls, rigid balls, plasticine, and other such accessories. The physical and kinematic parameters of the tools, such as, but not limited to: dimensions, mass, elasticity, diameter, perimeter, spring tension, are known. Together with either the above-mentioned remote position imaging and sensing methods, or by using the location signals transmitted by the hand wearables, the real time spatial co-ordinates of the hand and finger phalanges are detected, and the exerted forces can be accurately calculated based on the pre-known parameters, without a need for force sensors on the fingers or on the tools. The force measurement accuracy depends on the sensor ability to locate the fingers precisely. Higher tool flexibility improves the accuracy of the force measurement, since the measured motion will be larger. The calculated forces can then be used to assess the progress of the rehabilitation process. The system can be programmed to provide objective assessment regarding the subject's impairment status. In such a system, the forces are objectively calculated and assessed with respect to an impairment scale.

Depending on the nature of the tool or object being used for the assessment, the system should be able to detect either just the fingers' or finger parts' spatial locations, or a combination of the fingers' or finger parts' spatial locations and the fingers' or finger parts' angular orientations. In many uses, there may indeed only be a need for a single degree of freedom, i.e. one direction of motion, for each finger or finger part. The motion of each finger or finger part may be in a different direction, but each one of them will be approximately follow linear motion. Thus, for instance, if a clothes pin is being used for the exercise being performed, the thumb and the first finger of the subject will both move approximately along the same direction of motion, but in opposite directions. Therefore, only change in the spatial location of the two relevant sensors or imaged locations will need to be tracked, and there will be no need to measure change of orientation as the subject squeezes the clothes pin. In that case, the system measures only change in linear location of the sensors or imaged locations, and not orientation. On the other hand, use of some of the tools or objects may mandate determination of both the spatial position and the angular orientation of each of the tracked hand elements, such as the phalanges of the fingers being monitored. The common exercise of compression of a ball may be considered to be such a case, in which at least some of the phalanges of the fingers being monitored should be tracked by sensors or their imaged positions which can output the complete pose, i.e. the co-ordinate position in the six degrees of freedom, of the element being monitored, since each sensor on each finger phalange may be moving in a different direction. The complete pose determination will provide the maximum accuracy of hand element tracking, but it is to be understood that use of only linear position measurements, while less accurate, may be sufficient for many rehabilitation exercises, and that the presently described systems should thus be understood to include different levels of automatic sensor element tracking, depending on the specific exercise and accessory being used. It is to be understood that when reference is made to the tracking of the motion of sensors, this term is meant to include also the motion as tracked by signal processing of images of the part of the fingers or hand, the imaged item being regarded as the “sensor” for the motion.

A method is further described in which the system can be used with gaming applications that make use of the location sensors, whether wearable or not, and whether transmitting their location information, or determining it from imaging methods, in order to calculate forces as defined above. This application enables integration of the exercises with games, a process which encourages the subject's motivation for the rehabilitation tasks. Game parameters and accessories can be adjusted by rehabilitation advancement, required hand movements, required forces, speed of action, a pre-planned rehabilitation plan and more. All the parameters can be adjusted to the patient's specific rehabilitation needs, progress and plan. Additionally, a database can be generated from collecting data based on the multiple system users. Optimized occupational therapy rehabilitation plans can be generated based on analysis of this collected data.

Several aspects for using the system and methods of this disclosure to apply to video games includes the following possibilities:
1. The patient and/or the therapist can track performance and progress in real-time on the screen, by demonstrating indicators related to the force exerted, and doing this for specific digits separately or for all of them.
2. A camera can serve as a mouse or a joystick and predefined exercises make the game work. In this case there is no need for designated occupational therapy (O.T.) games, the therapist defines the movements that need to be performed to make the game operate, and the patient does the exercise on games he plays at home.
3. Tracking performance and improvement, and building a database for optimizing the therapy based on Big Data analysis of the collected data.
4. Adaptable gaming based on patient performance and abilities determined during the game and the pre-defined exercise goals by the therapist.
5. Use of augmented reality to incorporate objects with known properties to the gaming.

There is thus provided in accordance with an exemplary implementation of the devices described in this disclosure, a system for use in hand rehabilitation of a subject, the system comprising:
(i) a position sensing system adapted to determine the spatial location and optionally the angular orientation of parts of the hand of the subject, and
(ii) a controller adapted to obtain information from the position sensing system regarding the change in position or the rate of change in position of at least one part of the hand of the subject, while the hand of the subject is handling at least one accessory, the at least one accessory having at least one known characteristic relating to the force required to actuate the accessory,
   wherein the controller uses the position information and the at least one known characteristic of the accessory to enable the system to output information regarding the hand rehabilitation of the subject, without the need for force sensors on any part of the hand of the subject or on any of the at least one accessory.

In such an above described system, the at least one characteristic may be at least one of a dimension or an elasto-mechanical property of the at least one accessory. In such a case, the elasto-mechanical property of the at least one accessory may be its mechanical stiffness.

In yet further implementations of the previously described systems, the at least one characteristic may be the geometrical dimensions of the at least one accessory.

Furthermore, the position sensing system may comprise at least one position-indicating sensor mounted on the part of the hand of the subject, the position-indicating sensor being configured to remotely transmit location signals to the controller. The position-indicating sensor may be mounted on a wearable element to be worn on the part of the hand of the subject whose pose is to be monitored. Alternatively, the position-indicating sensor may be a marker mounted directly on the part of the hand of the subject whose pose is to be monitored.

According to yet other implementations of the above described systems, other than those comprising position-indicating sensors mounted on the part of the hand of the subject, the position sensing system may comprise at least one remote camera adapted to generate images of the part of the hand of the subject or of a marker attached to the part of the hand of the subject, such that image processing of the images enables the position of the part of the hand to be determined. Alternatively, the position may be at least the spatial location of the part of the hand of the subject, and optionally also the angular orientation of the part of the hand of the subject.

In any of the above described systems, the part of the hand of the subject may comprise at least one of an index, a phalange of an index, or the tip an index. The index may be either of a finger or a thumb.

Finally in any of those systems, the controller may further be adapted to use the calculated forces to assess the level of the hand rehabilitation on an impairment scale.

There is further provided according to yet another implementation of the systems of the present application, a system for use in hand rehabilitation of a subject, comprising:
(i) a position sensing system adapted to determine the spatial location and optionally the angular orientation of parts of the hand of the subject, and
(ii) a controller adapted to compare the known geometrical dimensions of at least one accessory adapted to be used for exercising of the hand, with the determined spatial location and angular orientation of at least one part of the hand of the subject, when the hand is handling the at least one accessory, such that rehabilitation of the hand can be assessed.

In yet another implementation of the systems of the present application, there is even further provided a system for use in hand rehabilitation of a subject, the system comprising:
(i) an arrangement for determining at least one of the spatial location and angular orientation of at least one part of the hand of the subject, and
(ii) a controller adapted to obtain information regarding the rehabilitation process by utilizing:
   (a) at least one of the change in at least one of the spatial location and angular orientation, or the rate of change in at least one of the spatial location and angular orientation of at least one part of the hand of the subject, while using at least one accessory adapted for exercising of the hand, the at least one accessory having at least one characteristic relating to the force required to actuate the accessory, and
   (b) at least one characteristic of the at least one accessory.

In such a system, the arrangement may comprise a remote imaging system adapted to generate images of the at least one part of the hand of the subject. The remote imaging system may be adapted to generate images of a marker attached to the at least one part of the hand of the subject. In either of these cases, the at least one characteristic of the at least one accessory may be the mechanical stiffness of the at least one accessory. Alternatively, the at least one characteristic of the at least one accessory may be the geometrical dimensions of the at least one accessory.

### BRIEF DESCRIPTION OF THE DRAWINGS

FIG. 1A shows the components of the system, including wearable sensors able to remotely transmit exact location signals;

FIG. 1B shows typical rehabilitation toys/tools with known physical, geometric and kinematic parameters, intended for handling by the hand of the subject in order to determine the forces applied;

FIG. 1C shows a computerized system which processes the signals and provides rehabilitation training based on gaming applications;

FIG. 2A shows yet another embodiment in which the hand positions may be remotely located without the need to use any wearable sensors, by using one or more cameras for tracking movement of the hand parts;

FIG. 2B shows a subject's hand with highly visible markers on hand or finger parts, such that their positions can be more readily or more accurately determined by the camera or cameras of FIG. 2A; and

FIG. 3 shows schematically how the data from the subject's exercise with an accessory can be used by the system, whether using the sensors of FIG. 1A or the camera system of FIG. 2A, to determine the muscle strength or another characteristic of the hand motion achieved.

### DETAILED DESCRIPTION

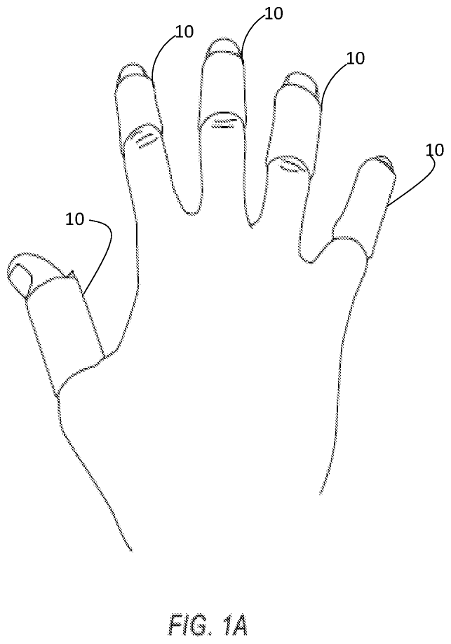

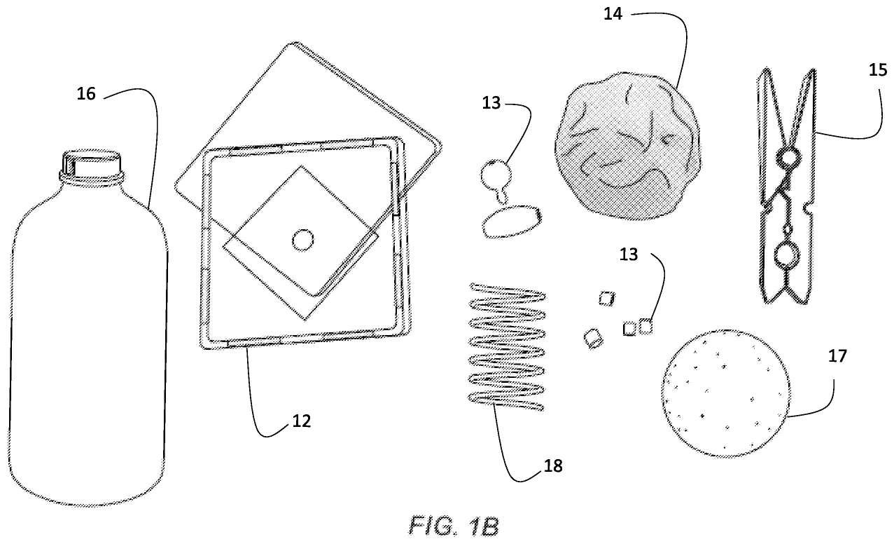

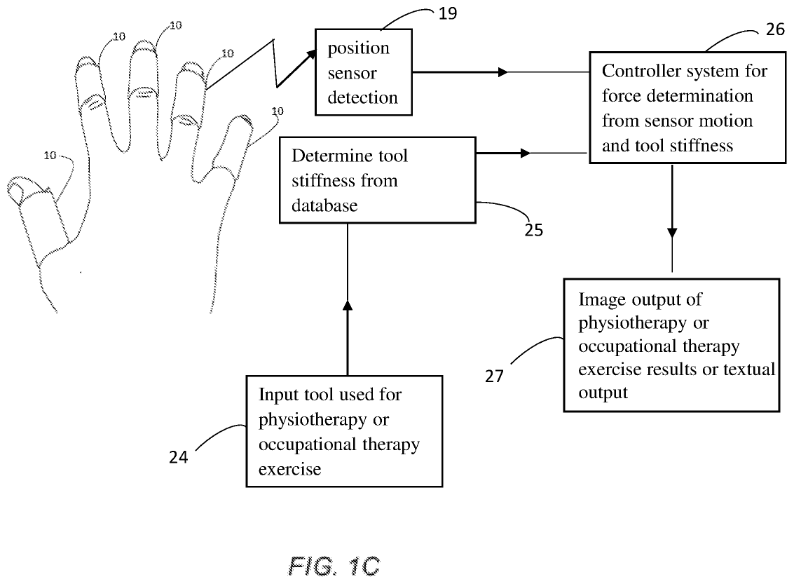

Reference is first made to FIG. 1A, which shows wearable sensor elements 10 of the proposed system, which are able to transmit at least one of location and orientation signals to a remote control system, by use of an electronic transmission device (not shown in FIG. 1A) mounted in the wearable sensor elements 10. The wearable wireless sensors can make use of, but are not limited to, the following technologies: inertial sensors, electromagnetic sensors, optical sensors, pressure sensors, touchpad sensors, electromyographic (EMG) sensors. Since the sensors generally require a receiver in a remote controller base station, which interrogates the signals received from the sensor elements in order to determine at least one of the position and orientation (pose) information which the sensor is providing, these sensor elements act as indicators of the pose of the element, which, together with the base station control system, provide the pose sensing. However, as is conventionally used, the term pose sensors or location sensors is used in this disclosure to describe an element which sends signals providing information to a controller about the position, with or without the orientation, of the element. Thus, the terms position indicating sensor, or pose sensor or position sensor, or similar terms using the word element, may have been used in this disclosure interchangeably, but all relate to the same position indicating element.

The above-mentioned spatial location sensors 10 should be located in a manner that permits movement in all degrees of freedom of the hand and fingers and enables position detection of each of the phalanges, without interfering with the hand's movement, thus allowing active patient rehabilitation. In this respect, such spatial location sensors show a significant advantage over the use of force sensors, which, besides being more costly, generally will impede the hand movements. The presently described spatial location sensors can be worn within an elastic glove. They can be worn as separate sensors on each finger and thumb, as illustrated in the example of FIG. 1A. It should be emphasized that, although the sensing can be performed on any part of the hand or the fingers, the following description of the system and method of the present disclosure, uses the common example of sensing the position of the finger tips as the sense hand/finger position. It is to be understood however, that this is merely an example of the position sensing use of the presently described system and is not intended to limit the scope in this respect of the invention to the exemplary fingertip position shown in some parts of the detailed description.

The location sensors can also be worn on each of the finger and thumb phalanges or in any setting suitable for the rehabilitation pre-plan. It is further emphasized that in one embodiment the sensors can be worn by a patient performing occupational therapy with a splint.

In such systems, as opposed to prior art systems, force sensors are not required for measuring the forces exerted by the hand and finger muscles. Such forces measured by the present system may include, but are not limited to, pressure, rotation and kneading forces. These forces are present in spatial movements conducted in daily activities or in a rehabilitation process, such as, but not limited to, rotation, abduction, adduction, opposition, pinching, gripping, flexion and extension. Such measurements can provide an objective assessment regarding the patient capabilities and the recommended rehabilitation plan.

The system can measure movement, and can calculate forces in all degrees of freedom, as opposed, for example, to existing in-use rehabilitation exercise glove-based products, such as for example the “Sinfonia” “Hand Tutor” or “Amadeo” systems, and other prior art systems that are limited to measuring hand or finger rotations and lateral motions.

In yet another aspect, the controller of the system can provide gaming applications through computer or mobile applications. The games are based on interactive tracking of movements and motions exerted by the patient's hand and are intended to increase motivation and involvement. The gaming applications can further be programmed to match a pre-defined rehabilitation plan, or to monitor real-time changes in the rehabilitation plan, based on patient improvement. In yet an additional aspect, the system can adaptively and automatically adjust exercise difficulty according to the patient's capabilities and/or real time performance while playing. The software detects the maximum or minimum forces exerted by the patient while playing, and sets gaming goals to meet the rehabilitation exercise requirements.

The system can be applied to one or both hands of the patient or the therapist, depending on the patient's clinical situation. In one embodiment, the healthy hand can serve as a reference for assessing impairment and improvement during the rehabilitation process, by measuring the forces exerted by the healthy hand in the same exercises as those performed by the impaired hand. The therapist can serve as a guide for the exercises.

Additionally, the therapist can focus on specific digit(s) and exercise(s) and show the patient in real time his/her progress according to pre-defined baseline and performance indicators. The system can also record and simulate the patient hand and/or finger movement to detect improvement over time or during a training session, in different rehabilitation parameters.

Reference is now made to FIG. 1B which shows a variety of occupational therapy tools for use with the system. In FIG. 1B, there are shown separable magnets 12, large and small plastic beads 13, theraputty 14, cloth pins 15, plastic bottles 16, sponge balls 17, a spring 18, rigid balls, plasticine (play dough), and other such accessories. The physical and kinematic parameters of the tools, such as, but not limited to, dimensions, mass, elasticity, diameter, perimeter, and spring stiffness, are known. Together with the above-mentioned methods using remote sensors or image signal processing, the exerted forces can be accurately calculated based on the pre-known parameters, without a need for force sensors on the fingers or in the tools. The force measurement accuracy depends on the sensor ability to locate the fingers precisely. Higher tool flexibility, if that is commensurate with the needs of the therapeutic treatment, improves the force measurement, since the movement generated is larger. The system can utilize any occupational therapy tool or toy provided its geometrical, physical and kinematic parameters are known. Moreover, the system can accurately calculate success in performing a pre-defined task such as picking a specific object, by knowing the object's geometrical parameters and positioning of the hand, fingers and thumb.

Such systems can be readily used at a remote setting such as the patient home for home training, with tele-access to medical staff. The patient may be guided offline or in real-time while practicing at home to make sure that the training is performed correctly, for instance by imitating the motions of the therapist.

The finger position sensors, whether wearable or remote, may send the data of the finger position to a computing or control system for implementing the methods of the present disclosure. Reference is now made to FIG. 1C, which shows schematically, an exemplary system and method which can be used in order to implement hand rehabilitation using the above described accessories and system. Prior to performing a physiotherapy or occupational therapy task, the object held or manipulated by the subject's hand, is input 24 to the system, either by the physiotherapist or another user. Additionally, if the system includes image recognition features that enables recognition of the object being held by the subject, input of the object to the system can be done automatically. Once the object or tool has been input into the system in step 24, a database of physiotherapy tools and their elasto-mechanical properties can then input in step 25, the known characteristics of the object held or manipulated by the subject's hand, such as its dimensions, shape, mass, rigidity, or any other elasto-mechanical properties, into the system. Alternatively, the physiotherapist or another user can input the characteristics directly, if known to them, without need to access the data base. The fingertip and phalange locations are known for continuously tracked in step 19, either from the data transmitted by the finger sensors 10, or as determined by image processing by a remote camera system, of an image of the position of the fingers, or of a marker on the fingers. Information about the finger positions is input into the controller or computer system 26, together with the information about the form and stiffness characteristics of the physiotherapy or occupational therapy tool being used to perform the exercise. The force used by the subject to perform the exercise is calculated, together with any other information that may be useful in determining the progress of the subject's rehabilitation, such as assessing the clinical impairment, like weakness in specific digits. In practice, with the object held by the hand, the computer can recognize finger locations at the object circumference by image processing of the images generated. From this point on, any relative change of the finger location can be attributed to the changing of the shape of the held object, and is detected by the position sensors or the remote camera(s) and input to the computer all control system 26. Since the flexibility of the object is a parameter now known to the computer system, the finger force can be calculated, for instance, by a Hooks law calculation. Finally, the generated information is output to a display device 27, either in graphic form, or as textual information.

In one embodiment, the accessories are equipped with identification fiducial markers, not shown in FIG. 1B, that identify which object is being used, and which may also identify its 3-dimensional position in space, such as by use of imaging cameras or any other location determining system. Based on the object location it is possible to relate the fingertip location to the object's configuration in space, and hence determine if the subject successfully grasps a specific object. In another embodiment the geometrical dimensions of the accessories are known, and based on a comparison of the measured fingertip locations with the known dimensions of the object, it is possible to determine if the subject has successfully grasped a specific object, without a need for fiducial markers. Gaming software may make use of augmented reality methods, while incorporating physical objects as part of the game. The known mechanical properties of the physical objects enable the defining of the force exerted during the game. Hence the therapist can adjust the game features to meet required exercise levels, based on the patient's training requirements.

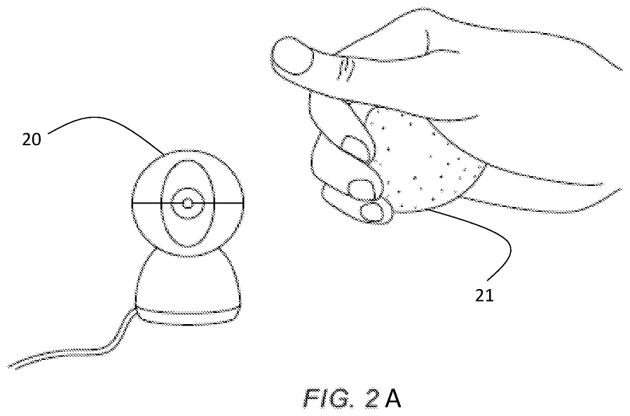

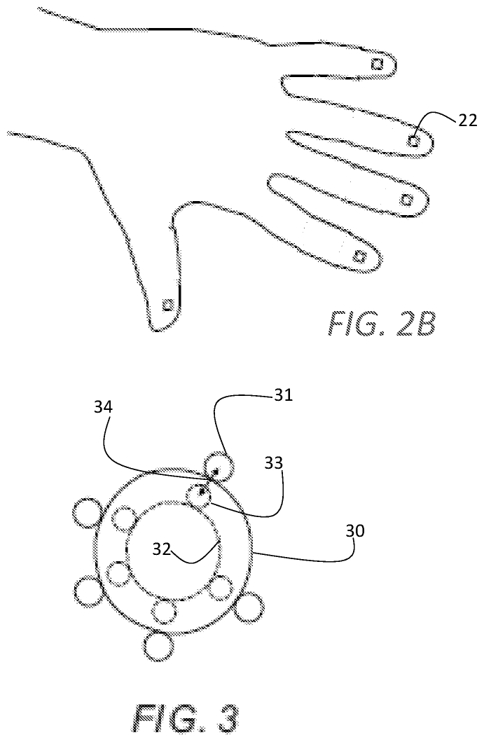

Reference is now made to FIG. 2A where, there is illustrated the embodiment in which the hand positions may be remotely located without the need to use any wearable sensors, such as, for example, by using one or more cameras 20 for tracking movement of the hand parts while using a physiotherapy accessory, shown in FIG. 2A as a sponge ball 21. As shown in FIG. 2B, in order to increase dimensional accuracy, it is also possible for the subject to wear highly visible markers 22 on hand or finger parts, such that their positions can be more readily or more accurately determined by the camera(s), and without the need for complex image processing programs to define the positions of the finger parts or hand from the images generated.

In these embodiments, image processing and analysis can also be made on a patient performing occupational therapy with a splint.

The remote camera(s) may be, but are not limited to, the “Leap Motion Controller” system, available from Ultraleap Ltd. of Bristol, UK or the “Intel RealSense” system camera devices available from Intel Inc. of Santa Clara, CA, USA.

Reference is now made to FIG. 3, which shows schematically how the data from a subject's exercise with an accessory can be used by the system, whether using the sensors of FIG. 1A or the camera system of FIG. 2A, to determine the muscle strength or another characteristic of the hand motion achieved. In FIG. 3, there is shown, for example, a schematical outline of a round manipulated object, such as a flexible ball 30, with a known dimension and with a known rigidity matrix. The position of the fingertips 31 around the circumference of the object 30 generates a circle that is defined by the detected positions of the finger tips, either using sensors on the fingers of the subject, as in FIG. 1A, such as RF sensors, electromagnetic sensors, ultrasound sensors, or any other sensor that determines the position in free space, or using a camera system, as in FIG. 2A, which determines the detected position of the finger tips or phalanges by image processing of the camera images, without the need for any wearables. Since the uncompressed radius of the object 30 is known, the extent to which the fingertips compress the ball 30 can be determined by the smaller radius 32 detected of the new detected positions 33 of the fingertips. From the rigidity matrix of the material of the ball 30 and the fingertip displacement 34, the force applied by the subject can be calculated, as a product of the displacement by the rigidity matrix.

It will be appreciated by those skilled in the art, in view of these teachings, that alternative embodiments may be implemented without deviating from the spirit or scope of the invention. For instance, sensors may be located on each of the fingers' phalanges, or in a glove embodiment, or camera imaging without wearable sensors can detect any of the fingers' phalanges. Additionally, different toys/tools from the exemplary toys or tools shown in this disclosure, can be used.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. Furthermore, it is appreciated by persons skilled in the art that the present invention is not limited by what has been particularly shown and described hereinabove. Rather the scope of the present invention includes both combinations and subcombinations of various features described hereinabove as well as variations and modifications thereto which would occur to a person of skill in the art upon reading the above description and which are not in the prior art.

1. A system for use in hand rehabilitation of a subject, the system comprising:
a position sensing system adapted to determine the spatial location and optionally the angular orientation of parts of the hand of the subject; and
a controller adapted to obtain information from the position sensing system regarding the change in position or the rate of change in position of at least one part of the hand of the subject, while the hand of the subject is handling at least one accessory, the at least one accessory having at least one known characteristic relating to the force required to actuate the accessory,
wherein the controller uses the position information and the at least one known characteristic of the accessory to enable the system to output information regarding the hand rehabilitation of the subject, without the need for force sensors on any part of the hand of the subject or on any of the at least one accessory.

2. A system according to claim 1, wherein the at least one known characteristic is at least one of a dimension or an elasto-mechanical property of the at least one accessory.

3. A system according to claim 2, wherein the elasto-mechanical property of the at least one accessory is its mechanical stiffness.

4. A system according to claim 1, wherein the at least one known characteristic is the geometrical dimensions of the at least one accessory.

5. A system according to claim 1 wherein the position sensing system comprises at least one position indicating sensor mounted on the part of the hand of the subject, the position indicating sensor being configured to remotely transmit location signals to the controller.

6. A system according to claim 1, wherein the position indicating sensor is mounted on a wearable element to be worn on the part of the hand of the subject whose pose is to be monitored.

7. A system according to claim 1, wherein the position indicating sensor is a marker mounted directly on the part of the hand of the subject whose pose is to be monitored.

8. A system according to claim 1, wherein the position sensing system comprises at least one remote camera adapted to generate images of the part of the hand of the subject or of a marker attached to the part of the hand of the subject, such that image processing of the images enables the position of the part of the hand to be determined.

9. A system according to claim 1, wherein the position comprises at least the spatial location of the part of the hand of the subject, and optionally also the angular orientation of the part of the hand of the subject.

10. A system according to claim 1, wherein the part of the hand of the subject comprises at least one of an index, a phalange of an index, or the tip of an index.

11. A system according to claim 10, wherein the index is either of a finger or a thumb.

12. A system according to claim 1, wherein the controller is further adapted to use the calculated forces to assess the level of the hand rehabilitation on an impairment scale.

13. A system for use in hand rehabilitation of a subject, comprising:
a position sensing system adapted to determine the spatial location and optionally the angular orientation of parts of the hand of the subject; and
a controller adapted to compare the known unstressed geometrical dimensions of at least one accessory adapted to be used for exercising of the hand, with the determined spatial location and angular orientation of at least one part of the hand of the subject, when the hand is handling the at least one accessory, such that rehabilitation of the hand can be assessed.

14. A system for use in hand rehabilitation of a subject, the system comprising:
an arrangement for determining at least one of the spatial location and angular orientation of at least one part of the hand of the subject; and
a controller adapted to obtain information regarding the rehabilitation process by utilizing:
(i) at least one of the change in at least one of the spatial location and angular orientation, or the rate of change in at least one of the spatial location and angular orientation of at least one part of the hand of the subject, while using at least one accessory adapted for exercising of the hand, the at least one accessory having at least one characteristic relating to the force required to actuate the accessory, and
(ii) at least one characteristic of the at least one accessory.

15. A system according to claim 14, wherein the arrangement comprises a remote imaging system adapted to generate images of the at least one part of the hand of the subject.

16. A system according to claim 14, wherein the remote imaging system is adapted to generate images of a marker attached to the at least one part of the hand of the subject.

17. A system according to claim 14, wherein the at least one characteristic of the at least one accessory is the mechanical stiffness of the at least one accessory.

18. A system according to claim 17, wherein the at least one characteristic of the at least one accessory is the unstressed geometrical dimensions of the at least one accessory.

19. A system according to claim 13, wherein the position sensing system comprises a remote imaging system adapted to generate images of the parts of the hand of the subject.

20. A system according to claim 13, wherein the at least one accessory has at least one characteristic relating to the force required to actuate the at least one accessory.
